(19) United States
(12) Patent Application Publication
(10) Pub. No.: US 2010/0189765 A1
(43) Pub. Date: Jul. 29, 2010

(54) IMPLANTABLE OCULAR DRUG DELIVERY DEVICE AND METHODS

(76) Inventors: Signe R. Erickson, Long Beach, CA (US); Jianbo Zhou, Rancho Santa Margarita, CA (US); Krip Punja, Irvine, CA (US); Nathan R.F. Beeley, Santa Barbara, CA (US)

Correspondence Address: Kagan Binder, PLLC, 221 Main Street North, Suite 200, Stillwater, MN, 55082, US

(21) Appl. No.: 12/625,705

(22) Filed: Nov. 25, 2009

Related U.S. Application Data
(60) Provisional application No. 61/200,294, filed on Nov. 26, 2008.

(51) Int. Cl. A61F 2/14; A61K 31/5575; A61P 27/02

(52) U.S. Cl. 424/427; 514/211.02

(57) ABSTRACT

The present invention provides implantable ocular drug delivery devices. Generally, the devices have a distal portion with a coil shaped body member and a proximal portion which contacts the sclera. In one aspect, the coil-shaped body member includes a unique configuration including two coiled portions with different pitches, which improves insertion of the device into the eye. In another aspect, the device has a proximal portion that includes a unique cap configuration having a concave distal face that improves stabilization of the device in the eye. In another aspect, the device includes a transitional portion between the cap and the coil-shaped body member that also improves stabilization of the device in the eye. The invention also provides methods for inserting the medical device into the eye, and methods for the treatment of an ocular condition.

### CROSS-REFERENCE TO RELATED APPLICATION

The present non-provisional patent application claims priority under 35 U.S.C. §119(e) from U.S. Provisional Patent Application having Ser. No. 61/200,294 filed on Nov. 26, 2008, and titled IMPLANTABLE OCULAR DRUG DELIVERY DEVICE AND METHODS, wherein the entirety of said provisional patent application is incorporated herein by reference.

### FIELD OF THE INVENTION

The invention relates to implantable intraocular medical devices which can be used to deliver bioactive agents to a treatment site in the eye.

### BACKGROUND OF THE INVENTION

Medical devices can be placed in the body for treatment of a medical condition, such as infection or disease. More recently, technologies have been developed that allow a drug to be released from the device to treat the condition. Some of these technologies involve the release of a drug from a polymeric coating formed on the surface of the device. Other technologies involve the release of the drug from an inner portion (e.g., lumen) of the device. Treatment may require release of the bioactive agent(s) over an extended period of time, such as weeks, months, or even years.

In addition, the surfaces of implantable medical devices are typically biocompatible and non-inflammatory, as well as durable, to allow for extended residence within the body. Implantable devices are also desirably manufactured in an economically viable and reproducible manner, and they are generally sterilizable using conventional methods.

In addition to challenges associated with drug delivery, there are also often challenges associated with the implantation of the device. Some devices, such as stents, can have particular structural features, such as being collapsible, that facilitate insertion of the device into a target site. Particular device configurations that improve placement of the device at a target location ultimately can also improve the drug delivery from the device. For example, such structural improvements desirably facilitate the insertion process and minimize tissue damage, improve the stability of the device at a target location, and enhance drug delivery via structural design.

Therapeutic agent delivery devices that are particularly suitable for delivery of a therapeutic agent to limited access regions, such as the vitreous chamber of the eye and inner ear are described in U.S. Pat. No. 6,719,750 (“Devices for Intraocular Drug Delivery,” Varner et al.) and U.S. Publication No. 2005/0019371 (“Controlled Release Bioactive Agent Delivery Device,” Anderson et al.).

Because description of the invention will involve treatment of the eye as an illustrative embodiment, basic anatomy of the eye will now be described in some detail with reference to FIG. 1, which illustrates a cross-sectional view of the eye. Beginning from the exterior of the eye, the structure of the eye includes the iris 2 that surrounds the pupil 3. The iris 2 is a circular muscle that controls the size of the pupil 3 to control the amount of light allowed to enter the eye. A transparent shell structure, the cornea 4, locates in front of the pupil 4 and the iris 2. Continuous with the cornea 4, and forming part of the supporting wall of the eyeball, is the sclera 5 (the white of the eye). The conjunctiva 6 is a clear mucous membrane covering the sclera 5. Within the eye is the lens 8, which is a transparent body located behind the iris 2. The lens 8 is suspended by ligaments 9 attached to the anterior portion of the ciliary body 10. The contraction or relaxation of these ligaments 9 as a consequence of ciliary muscle actions changes the shape of the lens 8, a process called accommodation, and allows a sharp image to be formed on the retina 11. Light rays are focused through the transparent cornea 4 and lens 8 upon the retina 11. The central point for image focus (the visual axis) in the human retina is the fovea 12. The optic nerve 13 is located opposite the lens 8.

There are three different layers of the eye, the external layer, formed by the sclera 5 and cornea 4; the intermediate layer, which is divided into two parts, namely the anterior (iris 2 and ciliary body 10) and posterior (the choroid 14); and the internal layer, or the sensory part of the eye, formed by the retina 11. The lens 8 divides the eye into the anterior segment (in front of the lens) and the posterior segment (behind the lens). More specifically, the eye is composed of three chambers of fluid: the anterior chamber (between the cornea 4 and the iris 2), the posterior chamber (between the iris 2 and the lens 8), and the vitreous chamber (between the lens 8 and the retina 11). The anterior chamber and posterior chamber are filled with aqueous humor whereas the vitreous chamber is filled with a more viscous fluid, the vitreous humor.

### SUMMARY OF THE INVENTION

The present invention is directed to medical devices which can be implanted in a portion of the inner eye and capable of releasing bioactive agent(s). These devices are referred to herein as “implantable ocular devices.” The invention is also directed to methods for inserting the implantable ocular devices into the eye, and methods for treating ocular conditions with a bioactive agent that is released from the device. In one aspect, the device is used in a method wherein a portion of the device is inserted into the posterior of the eye so it can release one or more bioactive agents into the vitreous.

Generally, the implantable ocular device includes a distal portion comprising a coil-shaped body member that is configured to be rotatably inserted through the scleral tissue in a corkscrew-like manner. Rotation causes the coil shaped body member (starting with the distal end) to move through the sclera until a substantial portion of the coiled body is located in the vitreous. In the vitreous, the body member releases one or more bioactive agents for the treatment of an ocular condition.

In some embodiments, the implantable ocular device can also include a proximal portion comprising a cap, and a transitional portion located between the coil-shaped body member and the cap. The distal face of the cap can mate against the outer surface of the eye and help stabilize the device. The transitional portion of the device can be in contact with the sclera, and can also help stabilize the device.

Generally, the present invention provides implantable ocular drug delivery devices with novel and inventive features that improve the implantation, drug delivery, and stabilization of the device when inserted in the eye. In one aspect of the invention, the device has a coil-shaped distal portion with a unique and inventive configuration that facilitates its implantation and drug delivery. In other aspects of the invention, the device has a proximal portion with a unique and inventive configuration which facilitates stabilization of the device following implantation.

Accordingly, in one embodiment, the invention provides an ocular drug delivery device, including a proximal portion configured to contact the sclera of the eye, and a distal end having a coil-shaped body member comprising a first portion and a second portion. The first portion of the coil-shaped body member has a pitch that is greater than the second portion, and the second portion is proximal to the first portion (i.e., the second portion is closer to the proximal end). The device also includes a bioactive agent, which can be delivered from the distal portion of the device.

The unique coil-shaped body member (with first and second portions having different pitches) provides advantages for the implantation and function of the device. For example, during the implantation procedure, and upon application of rotational movement to the device, the inventive configuration of the distal portion facilitates the penetration of the tip (distal end) of the device into the scleral tissue. By doing so, damage to the scleral tissue, which may be otherwise caused by rotation of the tip on the surface of the sclera without penetration of the tip, is avoided. Undesirable tissue responses, such as inflammation, can also be minimized.

The unique design of the coil-shaped body member can improve insertion and at the same time maintain a high loading capacity for one or more bioactive agents. The coil-shaped configuration provides an excellent way to achieve a high loading of drugs as provided by the large surface area (or volume) of the body member at the distal portion of the device. For example, bioactive agent can be present and releasable from a coating and/or a lumen of the coil-shaped portion. The design of the distal portion allows the length of the device to be limited along its longitudinal axis so its distal end does not enter the central visual field.

Other embodiments of the invention are directed to unique and inventive proximal portion designs that improve, in the least, stabilization of the device, and patient compliance. In these embodiments, the implantable ocular device includes a proximal portion with a cap and/or a transitional portion connecting the coil-shaped body member to the cap. During the insertion process, as the body member becomes fully inserted into the eye by rotation, the distal face of the cap contacts the outer surface of the eye and stabilizes the device in its inserted position.

Therefore, in another embodiment, the invention provides an ocular drug delivery device having a proximal portion comprising a cap having a distal face with a concave shape. Upon insertion of the device, the distal face of the cap having the concave shape becomes flush with the outer surface of the eye, which is convex. The concave shape of the distal face provides enhanced contact with the outer surface of the eye. With the device in the fully inserted position, the concave shape can improve stabilization of the device and minimize movement of the distal portion in the vitreous.

In another embodiment, the cap structure has a configuration that improves stabilization of the device by minimizing irritation to the outer eye. In this embodiment, the cap structure has a periphery comprising a rounded cross-sectional shape. The rounded shape reduces irritation to sclera and conjunctiva, which in turn can improve stabilization of the device by minimizing translational movement of device.

The transitional portion refers to the part of the device between the coil-shaped body member and the cap. The transitional portion is configured to improve the stabilization of the device when inserted in the eye. In particular, when fully inserted in the eye, the transitional portion is in contact with the scleral tissue. In some aspects, the invention provides a device including a transitional portion, which is a linear section that is parallel to the central axis of the device and having a length in the range of about 0.15 mm to about 0.3 mm. Distal to this linear section, the body member curves into the coil shape of the second portion of the coil-shaped body member. The short transitional portion slightly spaces the coil shaped body member away from the distal face of the cap. This spacing improves the placement and stabilization of the device in the eye by wedging scleral tissue into a groove created by the cap, the transitional portion, and the proximal end of the coil-shaped body member.

The portions of the device that improve stabilization are beneficial as they minimize tissue irritation and can result in the implanted device being more tolerable to a patient during the period of insertion.

In some embodiments, a primary function of the device is to deliver the bioactive agent(s) to a desired treatment site within the eye. Once the desired treatment of the eye has been accomplished, the device can be removed from the body. Moreover, embodiments of the invention provide a device that is minimally invasive such that risks and disadvantages associated with more invasive surgical techniques can be reduced.

### BRIEF DESCRIPTION OF THE DRAWINGS

FIG. 1 is a cross-sectional view of an eye.

FIG. 2a is a perspective view of an implantable ocular device as shown from the proximal end of the device, and FIG. 2b is a perspective view of an implantable ocular device as shown from the distal end of the device.

FIG. 3 is a cross-sectional, two-dimensional view of an implantable ocular device.

FIG. 4 is a view from the proximal end of the device without the cap.

FIG. 5 is a cross-sectional, two-dimensional view of an implantable ocular device.

FIG. 6 is a cross-sectional, two-dimensional view of an implantable ocular device, showing the transitions portion in greater detail.

FIG. 7a is a perspective view of the cap portion shown from the proximal end of the device, and FIG. 7b is a perspective view of the cap portion as shown from the distal end of the device.

FIG. 8 is a cross-sectional view of the cap portion of the device.

FIG. 9 is a cross-sectional, two-dimensional view of an implantable ocular device, shown inserted in a portion of the eye and traversing the scleral layer.

### DETAILED DESCRIPTION OF THE INVENTION

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

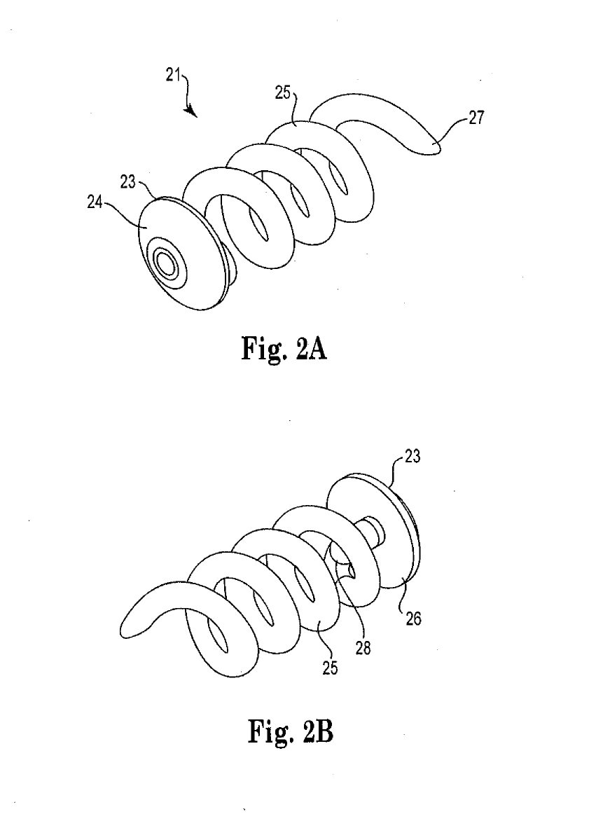

FIG. 2a shows an illustration of an exemplary implantable ocular device 21 of the present invention, with the proximal end being the closer end in view. The implantable ocular device 21 includes a cap 23, the proximal face 24 of which is shown, a distal portion having a coiled-shaped body member 25, and a distal end 27 that is sharpened. FIG. 2b shows an illustration of the exemplary implantable ocular device with the distal end being the closer end in view, and showing the distal face 26 of the cap 23, and the transitional portion 28 which is between the coiled-shaped body member 25 and the distal face of the cap 26.

A coiled shaped body member can be defined by distal and proximal ends of the coil. Generally, the coil-shaped body member follows a non-linear path around a central axis, which runs from the distal tip of the device to the proximal end of the coil at the transitional portion. As a general matter, a coil in the form of a helix follows a non-linear path continuously changing in direction, with the change in direction being constant. The change in direction being constant corresponds to a constant curvature and constant torsion of the helix. As such, in some embodiments, the device has a “helically-shaped” body member.

Generally, in a coil configuration, the individual rings of the coil rotate about the longitudinal axis. The overall coil can be substantially symmetrical about the longitudinal axis. Contemplated coils are composed of multiple rings that are substantially similar in circumference (as caused by a constant curvature) along the length, from proximal to distal, of the coil-shaped body member. Such individual rings can be concentric (that is, having a common axis, or being coaxial about the longitudinal axis) or eccentric (deviating from a circular path). According to these embodiments, the individual rings are noncontiguous along the body member length, thereby forming individual “ribs” at positions along the direction of extension of the body member. The curvature of the coil is measured as the arc of curvature relative to the central axis. Typically, the curvature of the first portion and the second portion of the coil shaped body member is the same.

The coil-shaped configuration of the body member provides an increased surface area (or volume) for delivery of a bioactive agent to an implantation site as compared to a linear device having the same length and/or width. This can provide advantages during use of the device, since this configuration allows a greater surface area to be provided in a smaller length and/or width of the device. For ocular applications, it can be desirable to limit the length of the device. For example, it is desirable to limit the length of implants in the eye to prevent the device from entering the central visual field of the eye and to minimize risk of damage to the eye tissues. By providing a body member that has at least a portion of the body member deviating from the direction of extension, the device of the invention has greater surface area for a bioactive agent-containing coating (and thus can provide more bioactive agent) per length of the device without having to make the cross section of the device, and thus the size of the insertion incision, larger.

As shown in FIGS. 2a and 2b the coil-shaped body member 25 follows a path from the proximal to the distal end that includes multiple rotations about a central axis of the device. A single rotation refers to a 360° turn in the body member. In many embodiments the coil-shaped body member has about three to about five full rotations (1080°-1800°), about three and a half to about four and a half full rotations, or about four full rotations. As shown in these figures, the coil-shaped body member follows a non-linear path to provide a configuration so that it is spaced from itself along its rotation. In other words, in many embodiments, the device has a configuration wherein the surface of the coil-shaped body member is not in contact with itself along its length.

The coil-shaped body member 25 as shown in FIGS. 2a and 2b is a left-handed helix and is implanted with clockwise rotation. Alternatively, the coil-shaped body member could have a right-handed helix. Insertion of body member with a right-handed helix would include counter-clockwise rotation.

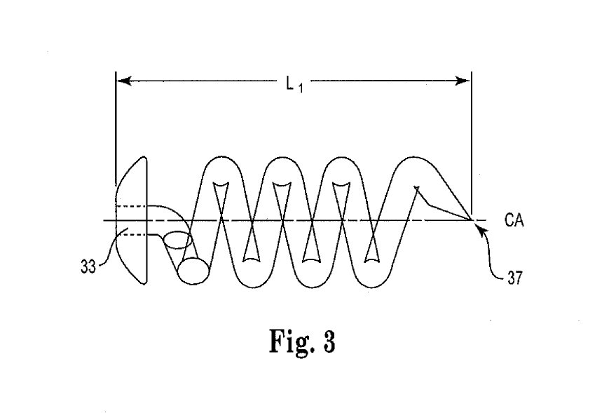

Referring to FIG. 3 (showing a cross-sectional view of the device), the device has a central axis (line CA) which is aligned with the center of the cap 33 and runs from the proximal to distal end 37 of the device. As measured along the central axis, the device can have an overall length (L₁) from the proximal end (proximal face of the cap) to the distal end (sharpened end of the coiled portion). The overall length L₁ of the implant can be limited to prevent the distal portion of the device from entering the central visual field of the eye and to minimize risk of damage to the eye tissues. Generally, in many embodiments, the overall length L₁ is less than about 1 cm. In many embodiments the overall length L₁ is in the range of about 2.5 mm to about 8.9 mm, more specifically in the range of about 4.1 mm to about 7.3 mm, or more specifically in the range of about 4.9 mm to about 6.5 mm. In exemplary embodiments, the overall length L₁ is about 5.7 mm, or about 6.0 mm.

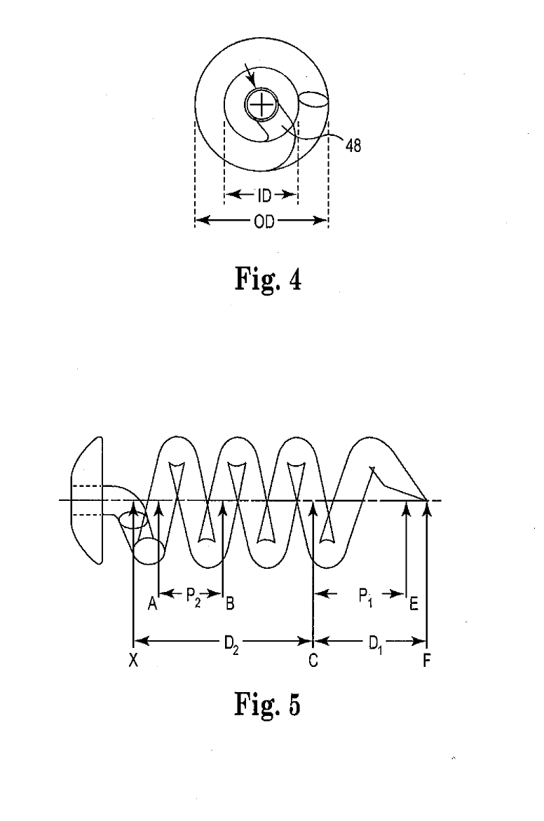

FIG. 4 shows a view of the transitional portion and coil-shaped body member of the device from the proximal end (without cap), looking down the central axis and showing an inner area of the. From this view, the inner area of the distal portion can be defined by an inner diameter (ID), which can also be referred to as the minor diameter. The inner diameter of the device can be uniform along the length of the coil-shaped distal portion, or can change along its length.

In many embodiments the inner diameter is in the range of about 0.43 mm to about 2.1 mm, more specifically in the range of about 0.85 mm to about 1.7 mm. In one exemplary embodiment, the inner diameter is about 1.28 mm.

Also shown in FIG. 4 is an outer diameter (OD) of the device, which can also be referred to as the “major diameter.” The outer diameter of the device can be uniform along the length of the coil-shaped distal portion, or can change along its length. In many embodiments the outer diameter is in the range of about 1.28 mm to about 2.19 mm.

Referring again to FIG. 4, a cross sectional shape of the body member is shown (referring to the transitional portion 48, at the point where the body member meets the distal face of the cap). The cross section shows the body member having a circular shape. In many aspects, the cross sectional shape of the body member is the same from beginning at the distal face of the cap (including the transitional portion) to near the distal end of the body member. For example, the cross sectional shape of the body member is substantially circular along its length. This is exemplified by a body member formed from a rod or wire, wherein the rod or wire is configured to have a coil shape.

In some aspects the cross section of the body member has a diameter in the range of about 0.38 mm to about 0.63 mm, or more specifically in the range of about 0.45 mm to about 0.55 mm. In one exemplary embodiment, the body member has a diameter of about 0.5 mm, or a diameter of about 0.4 mm.

The shape of a cross section of the body member can also be substantially circular, oval, or can be of another non-curved shape. For example, the shape of a cross section of the body member can be polygonal, such as square, rectangular, hexagonal, or octagonal, etc.

The body member also has a cross sectional area, which can be determined. In many aspects, the body member has a cross sectional area in the range of about 0.11 mm² to about 0.31 mm², or more specifically in the range of about 0.16 mm² to about 0.24 mm². In one exemplary embodiment, the cross sectional area of the body member is about 0.20 mm², or about 0.13 mm².

In one embodiment, the device comprises a coil-shaped body member comprising a first portion and a second portion, wherein the first portion of the coil-shaped body member has a pitch that is greater than the second portion, and the second portion is proximal to the first portion. That is, the second portion of the coil shaped body member, which has a pitch that is less than the first portion, is located between the proximal portion of the device (e.g., the cap) and a point along the coil shaped body member where the first portion begins.

Pitch refers to the distance, as measured along the central axis, between two points on the body member, the two points being separated by a full (360°) rotation of the coil. Pitch can also be measured, however, knowing the distance along the central axis for a partial rotation of the coil. As an example, referring to FIG. 5, the coil shaped body member has a pitch P₂ in the second portion of the coil-shaped portion measured from point A to point B.

The first portion (having the greater pitch) begins at a point on the body member wherein there is a change in the torsion of the coil. Torsion is the rate of change of the osculating plane of a space curve. For example, the torsion of the helix is 1/T=2πa/p, where p is the length of rod for one turn of the helix and a is the pitch length. In other words, while the second portion of the body member can have a coiled shape that follows a non linear path continuously changing in direction, with the change in direction being constant (a continuous curvature and torsion), the first portion (having the greater pitch) can begin at a point on the body member wherein there is a change in constancy of the directional change of the second portion. That is, the first portion of the body member can begin at a point where there is a change in the torsion along the coiled path. Starting at this point, and moving distally along the body member, the helix becomes elongated, accounting for the increase in pitch in the first portion. As an example, referring to FIG. 5, the change in torsion of the body member begins at point C, and the coil-shaped body member has a pitch Pᵢ in the first portion of the coil-shaped portion measured from point C to point E.

The first portion (having the greater pitch) can be less than one full rotation of the helix, one full rotation of the helix, or more than one full rotation of the helix. In many embodiments, the first portion comprises about one quarter to one and a half rotations, about one half to about one and a quarter rotations, or about three quarters to about one full rotation.

In some aspects the first portion (having the greater pitch) has a pitch (for example, P₁ of FIG. 5) in the range of about 1.2 mm to about 2 mm, more specifically in the range of about 1.4 mm to about 1.8 mm, or more specifically in the range of about 1.5 mm to about 1.7 mm. In one particular aspect, the first portion has a pitch of about 1.6 mm.

The first portion typically has a distance (for example, D₁ from point C to point F of FIG. 5) as measured along the central axis in the range of about 1.28 mm to about 2.14 mm, more specifically in the range of about 1.5 mm to about 1.93 mm, or more specifically in the range of about 1.61 mm to about 1.82 mm. In one particular aspect, the first portion is about 1.71 mm in length. In many aspects, the second portion has a length (e.g., point X to point C of FIG. 5) that is greater than a length of the first portion (for example, from point C to point F of FIG. 5).

The first portion can have a constant or non-constant torsion. In some cases the first portion has a non-constant torsion. For example, the torsion in the first portion can increase towards the distal end of the body member. In this case, the helix can become further elongated towards the distal end.

In some aspects the second portion has a pitch (for example, P₂ of FIG. 5) in the range of about 0.74 mm to about 1.23 mm, more specifically in the range of about 0.84 mm to about 1.10 mm, or more specifically in the range of about 0.91 mm to about 1.04 mm. In one particular aspect, the second portion has a pitch of about 0.98 mm.

The second portion typically has a distance D₂ as measured along the central axis (for example, from point X to point C of FIG. 5) in the range of about 2.12 mm to about 3.53 mm, more specifically in the range of about 2.47 mm to about 3.18 mm, or more specifically in the range of about 2.65 mm to about 3.0 mm. In one particular aspect, the second portion is about 2.82 mm in length.

In many embodiments, the second portion comprises about two to four full rotations, about two and a half to about three and a half full rotations, or about three full rotations.

The length of the coil-shaped body member along its non-linear path (referring to the length of the body member if the coil was stretched straight), from the proximal end of the transitional portion to the distal end of the coil-shaped portion, can also be determined.

The length of the body member can be calculated knowing the pitch (P₂) and the circumference (C₂) of the second portion, the number of full rotations of the body member in the second portion (N₂), and the pitch (P₁) and the circumference (C₁) of the first portion, and the number of full rotations of the body member in the first portion (N_{I}) according to the following formula:

D=((P₁²+C₁²)^{-0.5}*N₁)+((P₂²+C₂²)^{-0.5}*N₂)

Typically the length of the coil-shaped body member along its non-linear path is in the range of about 15 mm to about 25 mm, or more specifically in the range of about 17.5 mm to about 22.5 mm. In one exemplary embodiment, the length of the body member along its non-linear path is about 20 mm.

The surface area of the coil-shaped body member along its non-linear path, from the proximal end of the transitional portion to the distal end of the coil-shaped portion, can also be determined knowing the length of the body member along its non-linear path.

Typically the surface area of the coil-shaped body member is in the range of about 18.9 mm² to about 49.5 mm², or more specifically in the range of about 24.7 mm² to about 38.9 mm². In one exemplary embodiment, the surface area of the body member along its non-linear path is about 31.4 mm².

The distal end of the body member can have a shape suitable for insertion in a target area of the eye. In some aspects, the distal end is sharpened or pointed to pierce the scleral tissue during implantation of the device into the eye. A sharpened or pointed end of the device can be utilized to make an incision in the scleral tissue, rather than requiring separate equipment and/or procedures for making the incision site. In other words, a sharpened distal end provides a “self-starting” device for insertion into the eye and no conjunctival surgery or other device is necessary for initial penetration into the scleral tissue.

The sharpened distal end can be formed in the path of the first portion (having the greater pitch) of the coil-shaped body member. In other words, and in some aspects, the sharpened portion does not deviate, or does not substantially deviate, from the configuration of the first portion of the coil-shaped body member.

In some embodiments, the sharpened or pointed distal end can be formed by beveling the distal end of the first portion of the body member. As shown in FIG. 3, the distal end 37 of the coil-shaped body member is beveled. Relative to the path of the path of the coil-shaped body member at the distal end, the end can beveled at an angle (from the tip of the device) in the range of about 35° to about 55°, about 40° to about 50°, or about 45°. Measured relative to the central axis, the distal end can be beveled to an angle of about 10° or greater, or about 20°.

The beveling can create a flat surface near the distal end.

Beveling can provide a particularly sharp end useful for piercing the scleral tissue and driving the device into the eye. Such beveling is desirable as the coil-shape of the first portion can still be maintained, and the length of the device along its coil-shaped body member does not have to be compromised by the inclusion of any linear portion of significant length.

In general, materials used to fabricate the implantable ocular device are not particularly limited. In many aspects, the coil-shaped body member of the device is fabricated from a rigid, non-pliable material. The use of a rigid, non-pliable material can provide improved implant/explant characteristics to the device. Alternatively, the body member can be fabricated of a flexible material, so that small movements of the implantable ocular device will not be translated to the implantation site.

The coil-shaped body member can be fabricated partially or solely from metals. Suitable metals for the fabrication of the body member include platinum, gold, or tungsten, as well as other metals such as rhenium, palladium, rhodium, ruthenium, titanium, nickel, and alloys of these metals, such as stainless steel, titanium/nickel, nitinol alloys, and platinum/iridium alloys.

Ceramics such as silicon nitride, silicon carbide, zirconia, alumina, glass, silica, and sapphire, can also be used to fabricate the body member.

The body member can be fabricated partially or solely from plastic materials. Exemplary plastic materials include polyvinylchloride (PVC), polytetrafluoroethylene (PTFE), polyethersulfone (PES), polysulfone (PS), polypropylene (PP), polyethylene (PE), polyurethane (PU), polyetherimide (PEI), polycarbonate (PC), and polyetheretherketone (PEEK).

The coil-shaped body member can be solid, or alternatively, have one or more hallowed portions. A solid coil-shaped body member may be formed from a rod, whereas a hollow coil-shaped body member may be formed form a tube.

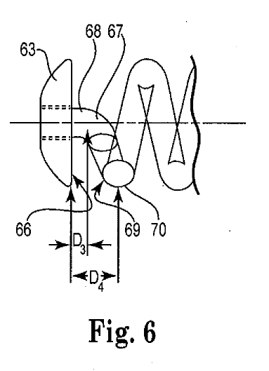

Referring to FIG. 6, the implantable ocular device can also include a transitional portion 68, which is located between the distal face 66 of the cap 63 and the proximal end 67 of the second portion of the coil-shaped body member. The transitional portion is configured to improve the stabilization of the device when inserted in the eye. In particular, when fully inserted in the eye, the transitional portion is in contact with the scleral tissue.

As shown in FIG. 6, the transitional portion 68 emanates from the distal face 66 of the cap 63 and is parallel to the central axis of the device for a very short distance, and then curves into the coil shape of the second portion of the coil-shaped body member. The short transitional portion slightly spaces the coil shaped body member away from the distal face of the cap. This spacing improves the placement and stabilization of the device in the eye.

The spacing between the distal face 66 of the cap and the proximal end 67 of the curved portion of the body member along the central axis of the device (shown as distance D₃) is in the range of about 0.15 mm to about 0.3 mm. The spacing between the distal face 66 of the cap and the outermost surface 70 of the first proximal rotation of the coil-shaped body member (shown as distance D₄) is in the range of about 0.5 mm to about 0.65 mm.

When fully inserted into the eye, the transitional portion is designed to stabilize the device by wedging scleral tissue between the distal face of the cap 66 and the surface 69 of the coil-shaped portion of the body member that faces the cap (the surface of the coil-shaped portion of the body member that is proximal to and opposite the distal face of the cap). The cap, transitional portion, and adjacent proximal surface of the coil shape body member form a groove that tightens upon the scleral tissue when the device is in place.

The proximal portion configuration with the unique transitional portion significantly improves stability of the device when fully inserted in the eye. The inserted device is less likely to experience unwanted movement, which may otherwise cause unwanted loosening of the positioning of the device, or may cause tissue irritation. The device does not require additional anchoring mechanisms (such as suturing) to the body tissues, as a result of the self-anchoring characteristics of the device itself.

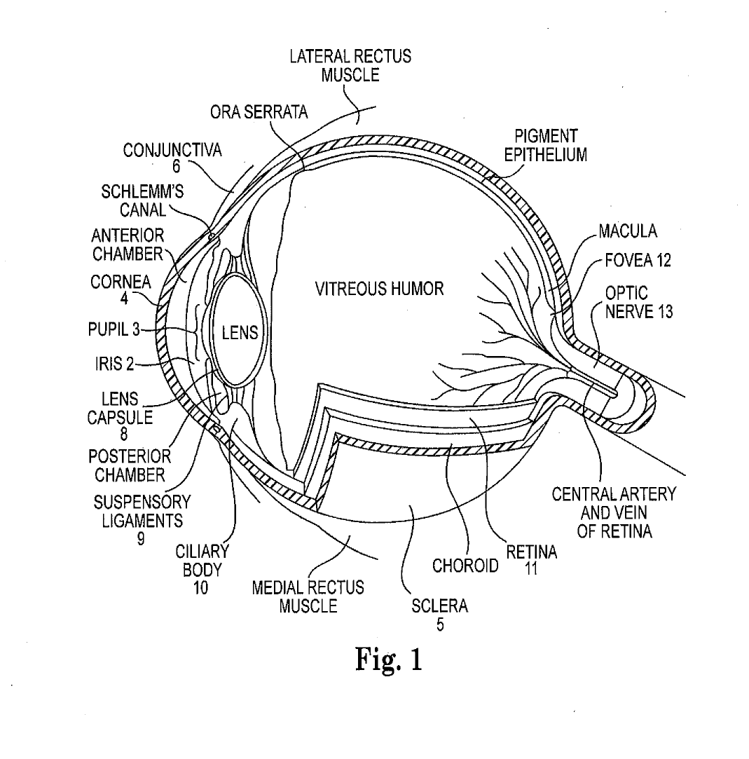

As shown in FIGS. 1a and 1b, the device can include a cap 23 which can assist in stabilization of the device once implanted in the body. Generally, the device is inserted through an opening in the scleral tissue until the distal face 26 of the cap 23 comes in contact with the exterior surface of the eye. The cap is designed to remain on the outside of the eye, and is sized so that it will not pass into the eye through the insertion site of the device. The cap can have an inventive configuration that improves stabilization of the device while at the same time minimizing tissue irritation, which may otherwise reduce patient compliance when the device is inserted into the eye. Desirably, the cap is configured and sized to be thin (as measured from the proximal 24 to the distal face 26 of the cap).

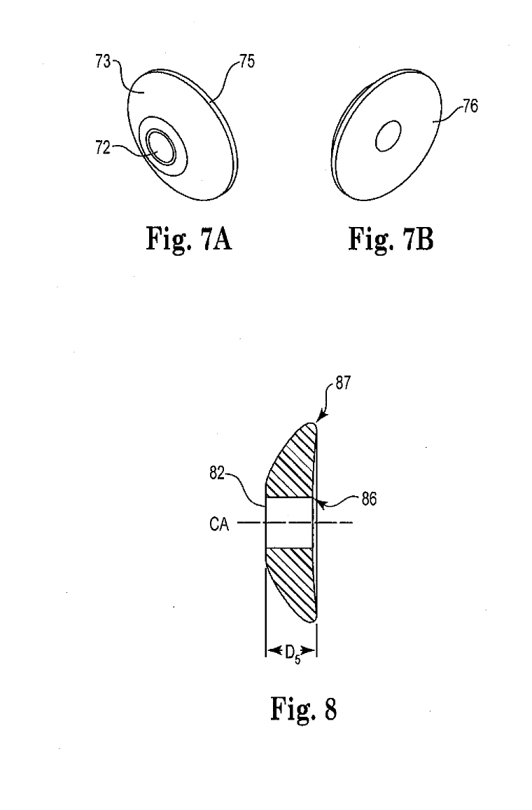

Referring to FIGS. 7a and 7b (showing the cap apart from the transitional portion and coil-shaped body member of the device), the cap is shown having a circular shape. However, the cap may have other non-circular shapes, such as oval, irregular curved shapes, and polygonal shapes. If such non-circular shapes are used, it is desirable that the periphery does not have sharp edges. Preferably the periphery 75 is curved or rounded. As shown in FIGS. 7a and 7b, a curved or rounded periphery can minimize tissue irritation and therefore improve patient compliance. In some aspects the cap, the periphery (e.g., the circumference if the cap has a circular shape) is in the range of about 4.52 mm to about 7.54 mm, about 5.28 mm to about 6.78 mm, or about 5.65 mm to about 6.41 mm. In one embodiment, the cap has a periphery of about 6.03 mm.

As shown in FIG. 7a, the proximal face of the cap can have a flat surface 72, and a curved surface 73. The flat surface 72 can be towards the center of the cap, and the curved surface 73 can be towards the periphery 75 of the cap. As such, in many aspects the cap is thicker near its middle, and thinner towards its periphery 75. The curved surface can have a constant curvature, or can have a non-constant curvature

The cap can taper from a maximal thickness near its center, to a minimal thickness near the periphery. For example, referring to FIG. 8, which shows the cap in cross section, as measured in the center of the cap (along the central axis) the thickness (distance D₅) can be in the range of about 0.25 mm to about 0.64 mm, and more specifically in the range of about 0.38 mm to about 0.51 mm. In some cases the cap has a constant thickness over a central portion of the cap.

This can provide the cap with a flat, or relatively flat surface 82 on its proximal face (72 in FIG. 7a). This flat surface can have an area in the range of about 0.245 mm² to about 0.405 mm², or more specifically about 0.285 mm² to about 0.365 mm². In one exemplary embodiment the cap has a flat surface with an area of about 0.325 mm².

In some aspects, the cap becomes thinner towards its periphery. For example, the cap can taper from a maximal thickness near the center of the device, to a minimal thickness near the periphery in the range of, for example, about 0.075 mm to about 0.175 mm, and more specifically in the range of about 0.10 mm to about 0.15 mm.

In some aspects, the cap has a curved peripheral edge 87, also shown in FIG. 8. The peripheral edge 87 of the cap is the transition from the proximal face to the distal face of the cap. The peripheral edge of the cap can have a curved surface that is rounded. When the device is implanted in the eye and the distal face of the cap is mated against the outer surface of the eye, the rounded peripheral end can also minimize tissue irritation and therefore improve patient compliance.

The distal face of the cap is in contact with the outer surface of the eye when the device is fully inserted into the eye. Therefore, the distal face of the cap can play a role in the stabilization of the device when inserted. In many aspects, the distal face of the cap is flatter than the proximal face of the cap. However, in some embodiments, the distal face of the cap includes a curved surface as show in FIG. 7b (76) and FIG. 8 (86). In some embodiments, the distal face of the cap has a concave surface, meaning that from the periphery of the cap, the surface curves inward. In an exemplary embodiment, the concave surface curves inward very slightly so that when the device is inserted into the eye the distal face intimately mates against the outer surface of the eye. In other words, the concave distal face of the cap fits with the convex shape of the outer surface of the eye. In some embodiments, the concavity (i.e., the depth of the distal face) is less than about 0.05 mm, and more typically about 0.032 mm

The cap can be fabricated from the same or different material as the transitional portion and/or the body member. In some embodiments, the cap can be fabricated from the same material as the body member. Alternatively, the cap can be fabricated from a material that is different from the body member.

The materials used to fabricate the cap are not particularly limited and include any of the materials previously described for fabrication of the coil-shaped body member. Generally, the materials are insoluble in body fluids and tissues with which the device comes in contact. Further, that the cap can be fabricated of a material that does not cause irritation to the portion of the body that it contacts (such as the area at and surrounding the incision site). For example, when the device is implanted into the eye, the cap is desirably fabricated from a material that does not cause irritation to the portion of the eye that it contacts. As such, materials for this particular embodiment include, by way of example, various polymers (such as silicone elastomers and rubbers, polyolefins, polyurethanes, acrylates, polycarbonates, polyamides, polyimides, polyesters, polysulfones, and the like), as well as metals (such as those described previously for the body member).

The cap can be fabricated separately from the coil-shaped body member, and subsequently attached to the body member, using any suitable attachment mechanism (such as, for example, suitable adhesives or soldering materials). For example, the cap can be fabricated to include an aperture, into which the body member is placed and thereafter soldered, welded, or otherwise attached. In alternative embodiments, the cap and body member are fabricated as a unitary piece, for example, utilizing a mold that includes both components (the body member and cap) of the device. The precise method of fabricating the device can be chosen depending upon such factors as availability of materials and equipment for forming the components of the device.

In another embodiment, the surface area of the coil-shaped body member can be increased by including surface configurations. Any suitable type of surface configuration can be provided to the body member, such as, for example, dimples, pores, raised portions (such as ridges or grooves), indented portions, and the like. Surface configuration can be introduced by roughening the surface of the material used to fabricate the body member. The surface of the body member can be roughened using mechanical techniques (such as mechanical roughening utilizing such material as 50 μm silica), chemical techniques, etching techniques, or other known methods. In other embodiments, surface introduced can be accomplished by utilizing a porous material to fabricate the body member. Alternatively, materials can be treated to provide pores in the material, utilizing methods well known in the art. In still further embodiments, surface configuration can be introduced by fabricating the body member of a machined material, for example, machined metal. The material can be machined to provide any suitable surface configuration as desired, including, for example, dimples, pockets, pores, and the like.

In some aspects of the invention, the device includes a coating on at least a portion of its surface. The coating can include a bioactive agent that is releasable from the coating following implantation of the device in the eye. Typically, the surface of the first and second portions of the coil-shaped body member of the device includes a coating. The transitional portion and the cap can also include a coating, however, this is optional.

A coating refers to one or more materials that are applied to the surface of the device. A bioactive agent releasing coating includes, in the least, a bioactive agent. More typically, a bioactive agent releasing coating includes a bioactive agent and at least one control-release component. In many aspects, the control release component is a polymeric material.

A coating can be formed using a “coating composition” which refers to the one or more materials used to form a coating on the surface of the device. A coating composition can include solids, such as bioactive agent, and one or more control-release component(s), and non-solids, such as one or more solvent(s), which can be used to dissolve or suspend the solid materials.

A “coated composition” or a “coating” refers to the solids material deposited on a surface of the implantable ocular device. The coated composition can be formed from one or more coating compositions, or in one or more layers. For example, if the coating is formed of multiple layers of coated material, the coated layers may also be described by “first coated layer”, “second coated layer”, and, if necessary, so forth. However, when describing a coating with multiple layers, whether a “first layer” is distal or proximal to the surface of the device will be understood in the context of the specific description of that coating.

For example, in some embodiments, the coated composition comprises at least two layers, wherein each layer comprises the same coated composition, or different coated compositions. In one such embodiment, a first layer having either bioactive agent alone, or bioactive agent(s) together with one or more of the polymers (first polymer and/or second polymer) is applied, after which one or more additional layers are applied, each with or without bioactive agent. These different layers, in turn, can cooperate in the resultant composite coating to provide an overall release profile having certain desired characteristics. This can be advantageous for the controlled release of bioactive agents having high molecular weights. The composition of individual layers of the coating can include any one or more of the following: one or more bioactive agents, a first polymer, and/or a second polymer, as desired.

A coated composition can be provided in contact with at least a portion of the coil-shaped body member of the device. In some embodiments, for example, it can be desirable to provide the coated composition in contact with the entire surface of the body member. Alternatively, the coated composition can be provided on a portion of the body member (such as, for example, an intermediate portion of the body member located between the proximal and distal ends thereof). In some embodiments, for example, it can be desirable to provide the coated composition in contact with a portion of the body member that does not include a sharp distal tip of the body member. This can be desirable, for example, to reduce risk of delamination of the coated composition at the sharp tip and/or to maintain the sharpness of the tip. The amount of the body member that is in contact with the coated composition can be determined by considering such factors as the amount of bioactive agent to be provided to the eye, the choice of coating material, risk of delamination of the coated composition, and the like. For example, in some embodiments, it can be desirable to provide the coated composition on portions of the body member other than the proximal and distal ends of the device, so as to reduce risk of delamination upon implant and/or explant of the device. Optionally, such delamination can also be minimized, in some embodiments, by providing a stepped coating thickness, such that the coating thickness decreases towards the proximal and/or distal ends of the body member. See, for example, the coating process described in U.S. Patent Application Publication No. 2005/0196424 (Chappa et al).

In still further optional embodiments, the device can be provided with a coated composition at distal and/or proximal portions that differs from the composition of the coating at the first and second portions of the coil shaped body member. One example of such an embodiment includes a body member having a lubricious coating at the distal end and/or proximal portion of the body member, with a different coated composition on the first and second portions of the coil shaped body member. One of skill in the art can determine the proportion and desired region(s) of body member to be coated.

Coated materials can be biocompatible with the body tissue or fluid that the device is place in contact with. As used herein, “biocompatible” means the ability of an object to be accepted by and to function in a recipient without eliciting a significant foreign body response (such as, for example, an immune or inflammatory response). For example, when used with reference to one or more of the polymers of the invention, biocompatible refers to the ability of the polymer (or polymers) to be accepted by and to function in its intended manner in a recipient.

In many aspects of the invention, the device includes a polymer-containing coating. One or more polymers can be included in the coating and provide control over the release of the bioactive agent from the device.

Polymeric materials useful for the present invention can be described in terms of molecular weight. Molecular weight (of a polymer preparation), as used herein, refers to the “weight average molecular weight” or M_{w}, which is an absolute method of measuring molecular weight and is particularly useful for measuring the molecular weight of a polymer preparation. The weight average molecular weight (M_{W}) can be defined by the following formula:

$${M}_{W} = \frac{{\sum }_{:} {N}_{i} {M}_{i}^{2}}{{\sum }_{:} {N}_{i} {M}_{i}}$$

wherein N represents the number of molecules of a polymer in the sample with a molecular weight of M, and Σᵢ is the sum of all NᵢMᵢ (species) in a preparation. The M_{w} can be measured using common techniques, such as light scattering or ultracentrifugation, gel permeation chromatography. Discussion of M_{w}, and other terms used to define the molecular weight of polymer preparations can be found in, for example, Allcock, H. R. and Lampe, F. W., Contemporary Polymer Chemistry; pg 271 (1990).

A coating formed on a surface of the device, or a matrix formed in a lumen of the device, can be stable, partially degradable or dissolvable, or fully degradable or dissolvable.

The term “degradable” as used herein with reference to polymers, shall refer to those natural or synthetic polymers that break down under physiological conditions (such as by enzymatic or non-enzymatic processes) into constituent components over a period of time. The terms “erodible”, “bioerodible”, “biodegradable” and “non-durable” shall be used herein interchangeably with the term “degradable”.

In some aspects, the device has a biostable coating or a biostable matrix formed from a biostable polymer. Exemplary biostable polymers include, but are not limited to, polymers of acrylates, vinyl polymers (such as ethylene vinyl acetates), urethanes, ethylene-based polymers (such as ethylene terephthalates and ethylene oxide), and silicones. Biostable polymers can be permeable to the bioactive agent, which can be released by diffusion through the polymeric coating or matrix. In some cases poly(ethylene-co-vinyl acetate) is used to form the biostable coating or matrix associated with the device.

In some aspects, the device includes a coating or a matrix formed from a poly(alkyl(meth)acrylate) and/or a poly(aromatic(meth)acrylate), wherein the designation “(meth)” includes such molecules in either the acrylic and/or methacrylic form (corresponding to the acrylates and/or methacrylates, respectively).

Exemplary poly(alkyl(meth)acrylates) include those with alkyl chain lengths from 2 to 8 carbons, inclusive, and with molecular weights from 50 kilodaltons to 900 kilodaltons. In one embodiment the polymeric material includes a poly(alkyl(meth)acrylate) with a molecular weight of from about 100 kilodaltons to about 1000 kilodaltons, from about 150 kilodaltons to about 500 kilodaltons, and more specifically from about 200 kilodaltons to about 400 kilodaltons. An example of a poly(alkyl(meth)acrylates) is poly(n-butyl methacrylate). Examples of other acrylate polymers are poly(n-butyl methacrylate-co-methyl methacrylate, with a monomer ratio of 3:1, poly(n-butyl methacrylate-co-isobutyl methacrylate, with a monomer ratio of 1:1 and poly(t-butyl methacrylate). Such polymers are available commercially (e.g., from Sigma-Aldrich, Milwaukee, Wis.) with molecular weights ranging from about 150 kilodaltons to about 350 kilodaltons, and with varying inherent viscosities, solubilities and forms (e.g., as slabs, granules, beads, crystals or powder).

Examples of suitable poly(aromatic(meth)acrylates) include poly(aryl(meth)acrylates), poly(aralkyl(meth)acrylates), poly(alkaryl(meth)acrylates), poly(aryloxyalkyl(meth)acrylates), and poly(alkoxyaryl(meth)acrylates).

Examples of suitable poly(aryl(meth)acrylates) include poly(9-anthracenyl methacrylate), poly(chlorophenyl acrylate), poly(methacryloxy-2-hydroxybenzophenone), poly(methacryloxybenzotriazole), poly(naphthyl acrylate), poly(naphthylmethacrylate), poly-4-nitrophenylacrylate, poly(pentachloro(bromo, fluoro) acrylate), poly(phenyl acrylate) and poly(phenyl methacrylate). Examples of suitable poly(aralkyl(meth)acrylates) include poly(benzyl acrylate), poly(benzyl methacrylate), poly(2-phenethyl acrylate), poly(2-phenethyl methacrylate) and poly(1-pyrenylmethyl methacrylate). Examples of suitable poly(alkaryl(meth)acrylates include poly(4-sec-butylphenyl methacrylate), poly(3-ethylphenyl acrylate), and poly(2-methyl-1-naphthyl methacrylate). Examples of suitable poly(aryloxyalkyl (meth)acrylates) include poly(phenoxyethyl acrylate), poly(phenoxyethyl methacrylate), and poly(polyethylene glycol phenyl ether acrylate) and poly(polyethylene glycol phenyl ether methacrylate) with varying polyethylene glycol molecular weights. Examples of suitable poly(alkoxyaryl(meth)acrylates) include poly(4-methoxyphenyl methacrylate), poly(2-ethoxyphenyl acrylate) and poly(2-methoxynaphthyl acrylate).

Acrylate or methacrylate monomers or polymers and/or their parent alcohols are commercially available from Sigma-Aldrich (Milwaukee, Wis.) or from Polysciences, Inc, (Warrington, Pa.).

The coating or matrix can also be formed by a mixture of two or more biostable polymers. For example, in one embodiment, the polymeric coating composition comprises poly(n-butyl)methacrylate (“pBMA”) and poly(ethylene-co-vinyl acetate) copolymers as the second polymer (“pEVA”). An exemplary absolute polymer concentration is in the range of about 0.05% to about 70% by weight of the coating composition. As used herein “absolute polymer concentration” refers to the total combined concentrations of first polymer and second polymer in the coating composition. In one embodiment, the coating composition comprises polyalkyl(meth)acrylate (such as poly(n-butyl)methacrylate) with a weight average molecular weight in the range of about 100 kilodaltons (kD) to about 1000 kD and a pEVA copolymer with a vinyl acetate content in the range of about 10% to about 90% by weight of the pEVA copolymer. In a particular embodiment, the polymer composition comprises polyalkyl(meth)acrylate (such as poly(n-butyl)methacrylate) with a molecular weight in the range of about 200 kD to about 500 kD and a pEVA copolymer with a vinyl acetate content in the range of about 30% to about 34% by weight. The concentration of the bioactive agent in the polymeric coating composition of this embodiment can be in the range of about 0.01% to about 90% by weight, based upon the weight of the final coating composition.

Exemplary mixtures of biostable polymers are described in U.S. Pat. No. 6,214,901 (Chudzik et al.) and U.S. Publication No. 2002/0188037 A1 (Chudzik et al.) (each commonly assigned to the assignee of the present invention). These documents describe polymer mixtures of poly(butylmethacrylate) (pBMA) and poly(ethylene-co-vinyl acetate) (pEVA).

Other useful mixtures of polymers that can be included in the coating or matrix are described in U.S. Publication No. 2004/0047911. This publication describes polymer blends that include poly(ethylene-co-methacrylate) and a polymer selected from the group consisting of a poly(vinyl alkylate), a poly(vinyl alkyl ether), a poly(vinyl acetal), a poly(alkyl and/or aryl methacrylate) or a poly(alkyl and/or aryl acrylate); not including pEVA.

The biostable polymeric material can also be a styrene copolymer, such as poly(styrene-isobutylene-styrene); the preparation of poly(styrene-isobutylene-styrene)-based coatings is described in, for example, U.S. Pat. No. 6,669,980.

In other forms of the present invention, the device includes a coating or a matrix comprising a biodegradable polymer. The coating or matrix can be formed from a biodegradable polymer that degrades in aqueous environments, such as by simple hydrolysis. The coating or matrix can be formed from a biodegradable polymer that is enzymatically degradable. For example, an enzymatically biodegradable polymer can be one that is degraded by enzymes produced by a mammalian body. Once broken down, the degradation products of these polymers are typically gradually absorbed or eliminated by the body.

Examples of classes of synthetic polymers that have been studied as biodegradable materials include polyesters, polyamides, polyurethanes, polyorthoesters, polycaprolactone (PCL), polyiminocarbonates, aliphatic carbonates, polyphosphazenes, polyanhydrides, and copolymers thereof. Specific examples of biodegradable materials that can be used in connection with the device of the invention include polylactide, polyglycolide, polydioxanone, poly(lactide-co-glycolide), poly(glycolide-co-polydioxanone), polyanhydrides, poly(glycolide-co-trimethylene carbonate), and poly(glycolide-co-caprolactone). Blends of these polymers with other biodegradable polymers can also be used. In many cases, release of a bioactive agent occurs as these polymers dissolve or degrade in situ.

Biodegradable polyetherester copolymers can be used. Generally speaking, the polyetherester copolymers are amphiphilic block copolymers that include hydrophilic (for example, a polyalkylene glycol, such as polyethylene glycol) and hydrophobic blocks (for example, polyethylene terephthalate). Examples of block copolymers include poly(ethylene glycol)-based and polybutylene terephthalate)-based blocks (PEG/PBT polymer). Examples of these types of multiblock copolymers are described in, for example, U.S. Pat. No. 5,980,948. PEG/PBT polymers are commercially available from Octoplus BV, under the trade designation PolyActive™.

Biodegradable copolymers having a biodegradable, segmented molecular architecture that includes at least two different ester linkages can also be used. The biodegradable polymers can be block copolymers (of the AB or ABA type) or segmented (also known as multiblock or random-block) copolymers of the (AB)ₙ type. These copolymers are formed in a two (or more) stage ring opening copolymerization using two (or more) cyclic ester monomers that form linkages in the copolymer with greatly different susceptibilities to transesterification. Examples of these polymers are described in, for example, in U.S. Pat. No. 5,252,701 (Jarrett et al., “Segmented Absorbable Copolymer”).

Other suitable biodegradable polymer materials include biodegradable terephthalate copolymers that include a phosphorus-containing linkage. Polymers having phosphoester linkages, called poly(phosphates), poly(phosphonates) and poly(phosphites), are known. See, for example, Penczek et al., Handbook of Polymer Synthesis, Chapter 17: “Phosphorus-Containing Polymers,” 1077-1132 (Hans R. Kricheldorf ed., 1992), as well as U.S. Pat. Nos. 6,153,212, 6,485,737, 6,322,797, 6,600,010, and 6,419,709. Biodegradable terephthalate polyesters can also be used that include a phosphoester linkage that is a phosphite. Suitable terephthalate polyester-polyphosphite copolymers are described, for example, in U.S. Pat. No. 6,419,709 (Mao et al., “Biodegradable Terephthalate Polyester-Poly(Phosphite) Compositions, Articles, and Methods of Using the Same). Biodegradable terephthalate polyester can also be used that include a phosphoester linkage that is a phosphonate. Suitable terephthalate polyester-poly(phosphonate) copolymers are described, for example, in U.S. Pat. Nos. 6,485,737 and 6,153,212 (Mao et al., “Biodegradable Terephthalate Polyester-Poly(Phosphonate) Compositions, Articles and Methods of Using the Same). Biodegradable terephthalate polyesters can be used that include a phosphoester linkage that is a phosphate. Suitable terephthalate polyester-poly(phosphate) copolymers are described, for example, in U.S. Pat. Nos. 6,322,797 and 6,600,010 (Mao et al., “Biodegradable Terephthalate Polyester-Poly(Phosphate) Polymers, Compositions, Articles, and Methods for Making and Using the Same).

Biodegradable polyhydric alcohol esters can also be used (See U.S. Pat. No. 6,592,895). This patent describes biodegradable star-shaped polymers that are made by esterifying polyhydric alcohols to provide acyl moieties originating from aliphatic homopolymer or copolymer polyesters. The biodegradable polymer can be a three-dimensional crosslinked polymer network containing hydrophobic and hydrophilic components that form a hydrogel with a crosslinked polymer structure, such as that described in U.S. Pat. No. 6,583,219. The hydrophobic component is a hydrophobic macromer with unsaturated group terminated ends, and the hydrophilic polymer is a dextran polysaccharide containing hydroxy groups that are reacted with unsaturated group introducing compounds. The components are convertible into a one-phase crosslinked polymer network structure by free radical polymerization.

The bioactive agent can also be delivered from a matrix comprising a poly(ester-amide) (PEA). Degradable poly(ester-amides) can include those formed from the monomers OH-x-OH, z, and COOH-y-COON, wherein x is alkyl, y is alkyl, and z is an alpha-amino acid. Examples of such alpha-amino acids are glycine, alanine, valine, leucine, isoleucine, norleucine, cysteine, methionine, phenylalanine, tyrosine, and tryptophan. The device can be associated with a matrix including a blend of two or more PEAs and a bioactive agent. Exemplary PEAs and blends are described in U.S. Pat. No. 6,703,040 (Katsarava, et al.)

Another biodegradable material comprises α-1,4 glucopyranose polymers. Some exemplary α-1,4 glucopyranose polymers that can be used to form the polymeric matrix are low molecular weight starch-derived polymers as described in commonly assigned under U.S. Pub. No. 2005/0255142, published Nov. 17, 2005, (Chudzik et al.) and U.S. Pub. No. 2007/0065481, published Mar. 22, 2007 (Chudzik et al.). These low molecular weight starch-derived polymers, as exemplified by amylose, maltodextrin, and polyalditol, comprise reactive groups, such as polymerizable groups, which can be activated to form a biodegradable matrix that includes bioactive agent.

The biodegradable polymer can comprise a polymer based upon α-amino acids (such as elastomeric copolyester amides or copolyester urethanes, as described in U.S. Pat. No. 6,503,538).

In other forms of the invention, the device includes a coating or a matrix comprising a biostable polymer and a biodegradable polymer.

In some cases the coating or matrix is formed from a composition wherein at least the biostable and biodegradable polymers are blended or dispersed in a common solvent or solvent system. Such a composition can be applied to a surface or filled with a lumen to form a coating or a matrix wherein the polymers are in mixture with each other. A suitable composition can be chosen based on the particular polymer components and solvent system used to solubilize or disperse the polymers.

In some aspects a coating or matrix is formed of a hydrophobic biostable polymer and biodegradable polymer comprising hydrophilic and hydrophobic segments. Combinations of biostable and biodegradable polymers, can be chosen based on the disclosure herein and those known in the art. An exemplary combination is of a poly((meth)acrylate), such as poly(butyl methacrylate) and biodegradable polyesters, such as a biodegradable poly(ether ester) multiblock copolymers based on poly(ethylene glycol) (PEG) and poly(butylene terephthalate) (PBT). Examples of these polymeric combinations are described in copending and commonly assigned U.S. Pub No. 2008/0038354.

As another example, biostable and biodegradable polymeric materials can be present in different coated layers on the surface of the device. For example, the device can include one coated layer formed of a biostable polymeric material, and a second coated layer formed of a biodegradable polymeric material. Bioactive agent can be present in one or both coated layers.

An additional coated layer can be present as a topcoat, which can cover one, or more coated polymeric layers that include a bioactive agent. Such topcoats can be used to modulate the release of a bioactive agent from the one or more layers underneath the topcoat. Topcoat materials can be biostable or biodegradable. In one aspect the coating can include an elution-controlling topcoat layer that comprises a poly(ethylene-co-vinyl acetate) copolymer. Such a top coat composition can be used for controlling the release rate of a hydrophilic bioactive agent from an undercoat. One topcoat composition uses a poly(ethylene-co-vinyl acetate) copolymer (pEVA) having a vinyl acetate concentration ranging from about 15% to about 35% vinyl acetate.

Examples of these pEVA polymeric topcoat compositions are described in copending and commonly assigned U.S. patent application Ser. No. 12/386,469, filed Apr. 17, 2009, and entitled COATING SYSTEMS FOR THE CONTROLLED DELIVERY OF HYDROPHILIC BIOACTIVE AGENTS (Hergenrother et al.)

An exemplary coating or matrix-forming composition can be prepared to include a solvent, one or more polymers dissolved or suspended in the solvent, and the bioactive agent or agents dispersed in the polymer/solvent mixture. The solvent is desirably one in which the polymers form a true solution. In some cases, the bioactive agent can either be soluble in the solvent or form a dispersion throughout the solvent. If the bioactive agent forms a dispersion, the coating composition and/or coating process may include the process of mixing or agitating the composition so the bioactive agent remains suspended in the composition.

In use, these embodiments do not require any mixing on the part of the user prior to application of the coating composition to the device. In some embodiments, the composition can provide a one-part system that can be applied to the device in one composition to form a coating, or that can be used to fill a lumen of the device. For example, U.S. Pat. No. 6,214,901 exemplifies the use of tetrahydrofuran (THF) as a solvent. While THF is suitable, and at times chosen for certain compositions, other solvents can be used in accordance with the invention as well, including, for example, alcohols (such as methanol, butanol, propanol, isopropanol, and the like), alkanes (such as halogenated or unhalogenated alkanes such as hexane and cyclohexane), amides (such as dimethylformamide), ethers (such as dioxolane), ketones (such as methylketone), aromatic compounds (such as toluene and xylene), acetonitrile, and esters (such as ethyl acetate).

In embodiments, the device is associated with a bioactive agent that is releasable from the device upon its implantation. For purposes of the description herein, reference will be made to “bioactive agent,” but it is understood that the use of the singular term does not limit the application of bioactive agents contemplated, and any number of bioactive agents can be provided using the teaching herein. Bioactive agents useful according to the invention include virtually any substance that possesses desirable therapeutic characteristics for application to the implantation site.

Examples of bioactive agents that can be associated with and releasable from the implantable ocular device of the invention are listed, but not limited to, those below

Steroids, including anti-inflammatory steroids and corticosteroids, can be associated with and releasable from the implantable ocular device. Exemplary anti-inflammatory steroids and corticosteroids include hydrocortisone, hydrocortisone acetate, dexamethasone 21-phosphate, fluocinolone, medrysone, methylprednisolone, prednisolone 21-phosphate, prednisolone acetate, fluoromethalone, betamethasone, and triamcinolone, or triamcinolone acetonide.

Various bioactive agents, which have anti-VEGF (vascular endothelial growth factor activity), such as VEGF-inhibitors or components which block production of VEGF, can be associated with and releasable from the implantable ocular device.

One type of VEGF-inhibitor is an anti-VEGF aptamer. Aptamers include DNA-based or RNA-based molecules and function similar to antibodies in that they are able to selectively bind to a target molecule, such as other nucleic acids and proteins. An example of a therapeutic aptamer is the pegylated anti-VEGF polynucleotide pegaptanib (Macugen™) for the treatment of age-related macular degeneration. Another type of anti-VEGF component is an anti-VEGF ribozyme. Enzymatic RNA molecules, known as ribozymes, can catalyze the cleavage and destruction of target RNA molecules. A ribozyme specific for the mRNA of FLT-1, known as Angiozyme™, which encodes a VEGF receptors in angiogenesis has been developed and shown to have potential for the treatment of advanced solid tumors.

Another type of VEGF-inhibitor is an anti-VEGF antibody or fragment thereof. Ranibizumab (Lucentis™) is an anti-vascular endothelial growth factor mAb fragment.

Antiproliferative agent can be associated with and releasable from the implantable ocular device. Exemplary antiproliferative agent include 13-cis retinoic acid, retinoic acid derivatives, 5-fluorouracil, taxol, rapamycin, analogues of rapamycin, tacrolimus, ABT-578, everolimus, paclitaxel, taxane, or vinorelbine.

Beta adrenergic agents can be associated with and releasable from the implantable ocular device. Exemplary beta adrenergic agents include isoproterenol, epinephrine, norepinephrine (agonists) and propranolol (antagonist).

Prostaglandins can be associated with and releasable from the implantable ocular device. Exemplary prostaglandins include PGE₂ or PGF₂.

Neuroprotective agents can be associated with and releasable from the implantable ocular device. Neuroprotective agents protect cells from excitotoxic damage. Such agents include N-methyl-D-aspartate (NMDA) antagonists, cytokines, and neurotrophic factors, more specifically coenzyme Q10, creatine, and minocycline

Exemplary neurotrophic factors include ciliary neurotrophic factor (CNTF) and glial cell-derived neurotrophic factor (GDNF).

Agonists of receptor tyrosine kinases can be associated with and releasable from the implantable ocular device. Exemplary receptor tyrosine kinases have been described in U.S. Pat. No. 5,919,813. In some aspects, the bioactive agent comprises a compound of formula I:

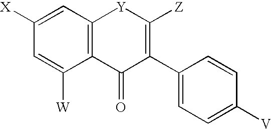

wherein V, W and X are selected from the group consisting of hydro, hydroxyl, alkoxy, halo, an ester, an ether, a carboxylic acid group, a pharmaceutically acceptable salt of a carboxylic acid group, and —SR, in which R is hydrogen or an alkyl group, and Y is selected from the group consisting of oxygen, sulfur, C(OH), and C═O, and Z is selected from the group consisting of hydro and C(O)OR₁, wherein R₁ is an alkyl. In some aspects, the alkoxy is a C₁-C₆ alkoxy. In some aspects, the halo is fluorine, chlorine or bromine. In some aspects, the ester is a C₁-C₆ ester. In some aspects, the ether is a C₁-C₆ ether. Pharmaceutically acceptable salts of the carboxylic acid group include sodium and potassium salts. In some aspects, the alkyl groups are C₁-C₆ alkyl groups. In some aspects, the protein tyrosine kinase pathway inhibitor is genistein.

The particular bioactive agent, or combination of bioactive agents, can be selected depending upon one or more of the following factors: the medical condition to be treated, the anticipated duration of treatment, the number and type of bioactive agents to be utilized, and the like.

The concentration of the bioactive agent in the coating composition or matrix-forming composition can be provided in the range of about 0.01% to about 90% by weight, based on the weight of the final composition.

In some aspects, the bioactive active agent is present in the coating composition or matrix-forming composition in an amount (percent by weight solids) in the range of about 4% to about 70%, or in an amount in the range of about 40% to about 60%, and in some exemplary compositions about 50%.

In some aspects the amount of bioactive agent in the coating composition or matrix-forming composition can be in the range of about 1 μg to about 10 mg, or about 100 μg to about 1500 μg, or about 300 μg to about 1000 μg.

In some aspects, the coating or matrix is formed having a weight-basis ratio of polymeric material to bioactive agent in the range of about 9:1 to about 3:7, or about 9:1 to about 4:6. The ratios are based on the total amounts of polymeric material and bioactive agent in the coating or matrix.

In some applications, additives can further be included with the bioactive agent and/or additional substance to be delivered to the implantation site. Examples of suitable additives include, but are not limited to, water, saline, dextrose, carriers, preservatives, stabilizing agents, wetting agents, emulsifying agents, excipients, and the like.

The coating or matrix-forming composition of the invention can be provided in any suitable form, for example, in the form of a true solution, or fluid or paste-like emulsion, mixture, dispersion, or blend. In many cases, the coating or matrix will generally result from the removal of solvents or other volatile components and/or other physical-chemical actions (for example, heating or illumination) affecting the coated composition in situ upon implantable ocular device surface.

The overall weight of the coated composition upon the surface of the device, or the matrix in one or more lumens of the device can be determined. For example, the device can be weighed before and after formation of the coating on the device, or formation of a matrix within a lumen. The weight attributable to the polymeric materials and bioactive agent can be determined, in combination or individually.

For example, the weight of the bioactive agent in the coating can be in the range of about 1 μg to about 5 mg of bioactive agent per cm² of the surface area of the implantable ocular device. In some embodiments, the surface area can comprise all or a portion of the body member of the device. In alternative embodiments, the surface area can comprise the body member and the cap of the device. In some cases, the weight of the coated composition attributable to the bioactive agent is in the range of about 0.01 mg to about 5 mg of bioactive agent per cm² of the surface area of the implantable ocular device. This quantity of bioactive agent is generally effective to provide adequate therapeutic effect under physiological conditions. As used herein, the surface area is the macroscopic surface area of the device.

In some embodiments, the surface of the body member can be pretreated prior to provision of the coating composition. Any suitable surface pretreatment commonly employed in coating implantable devices can be utilized in accordance with the invention, including, for example, treatment with silane, polyurethane, parylene, and the like. For example, Parylene C (commercially available from Union Carbide Corporation), one of the three primary variants of parylene, can be used to create a polymer layer on the surface of the implantable ocular device. Parylene C is a para-xylylene containing a substituted chlorine atom, which can be coated by delivering it in a vacuum environment at low pressure as a gaseous polymerizable monomer. The monomer condenses and polymerizes on substrates at room temperature, forming a matrix on the surface of the implantable ocular device. The coating thickness can be controlled by pressure, temperature, and the amount of monomer used. The parylene coating provides an inert, non-reactive barrier.

The coating composition can be applied to the implantable ocular device using any suitable method. For example, the coating composition can be applied by dipping, spraying, and other common methods for applying coating compositions to implantable devices. The suitability of the coating composition for use on a particular material, and in turn, the suitability of the coated composition, can be evaluated by those skilled in the art, given the present description.

In some aspects, the coating composition can be applied to the implantable ocular device utilizing an ultrasonic spray head as described in U.S. Publication Nos. 2005/0019371 (supra).

The coating composition is applied to the body member of the implantable ocular device surface in one or more applications. The method of applying the coating composition to the body member is typically governed by such factors as the geometry of the device and other process considerations. The coated composition can be subsequently dried by evaporation of the solvent. The drying process can be performed at any suitable temperature, (for example, room temperature or elevated temperature), and optionally with the assistance of vacuum.

In some modes of practice, a coating composition is applied to the body member under conditions of controlled relative humidity. As used herein, “relative humidity” is the ratio of the water vapor pressure (or water vapor content) to the saturation vapor pressure (or the maximum vapor content) at a given temperature of the air. According to some embodiments of the invention, the coating composition can be applied to the body member under conditions of increased or decreased relative humidity as compared to ambient humidity. When humidity is controlled at the time of applying the coating composition, the coating composition can be applied to the body member in a confined chamber or area adapted to provide a relative humidity that differs from ambient humidity. In one such embodiment, for instance, the coating composition is applied to the device under relative humidity controlled at a level in the range of about 0% to about 95% relative humidity (at a given temperature, in the range of about 15° C. to about 30° C.), and more specifically in the range of about 0% to about 50% relative humidity.

In some embodiments, the device has a coating with a thickness in the range of about 0.1 μm to about 100 μm, or in the range of about 5 μm to about 60 μm. This level of coating thickness is generally effective to provide a therapeutically effective amount of bioactive agent to the implantation site under physiological conditions. The final coating thickness can be varied, and at times be outside the ranges identified herein, depending upon such factors as the total amount of bioactive agent to be included in the coated composition, the type of bioactive agent, the number of bioactive agents to be included, the treatment course, the implantation site, and the like.

Thickness of the coated composition on the implantable ocular device can be assessed using any suitable techniques. For example, portions of the coated composition can be delaminated by freezing the coated implantable ocular device, for example, utilizing liquid nitrogen. The thickness at the edge of a delaminated portion can then be measured by optical microscopy. Other visualization techniques known in the art can also be utilized, such as microscopy techniques suitable for visualization of coatings having the thickness described herein of the invention.

In some embodiments, the cap can be provided with a polymeric coating composition. According to these particular embodiments, a polymeric coating composition provided in connection with the cap can be the same as, or different from, the polymeric coating composition provided in connection with the body member. For example, the particular bioactive agent included in the polymeric coating composition for the cap can be varied to provide a desired therapeutic effect at the incision site. Exemplary bioactive agents that could be desirable at the incision site include antimicrobial agents, anti-inflammatory agents, and the like, to reduce or otherwise control reaction of the body at the incision site. It will be readily apparent upon review of this disclosure that the first polymer and second polymer can also be selected for the polymeric coating composition provided in connection with the cap, to provide a desired polymeric coating composition specific for the cap, when desired.

In some aspects, the coil-shaped body member includes one or more lumens. The lumen(s) can extend along the length of the body member or only a portion of the length of the body member, as desired. In some aspects, the body member includes a single lumen that extends from the first portion to the second portion of the body member. The body member having a lumen can be formed from a tube that is formed into a coiled or helical configuration, such as shown in FIG. 3, having first and second portions.

The lumen(s) can serve as a delivery mechanism for delivery of a desired substance to the implantation site. The substance delivered via the lumen can comprise any of the bioactive agents described herein. The substance delivered via the lumen can be the same or different bioactive agent(s) from that included in a coating formed on a surface of the device.

The lumen can be loaded with a fill composition, which can be the bioactive agent itself, or the bioactive agent can be admixed with a material that modulates the release of the bioactive agent. For example, a fill composition for the lumen can contain a polymeric material that forms a polymeric matrix in the lumen and modulates the release of the bioactive agent out of the lumen. Polymeric materials useful for forming a coating, as described herein, can also be used to fill the lumen.

For the preparation of a device having a lumen, a fill composition can be delivered to the lumen through a port in the device, or more than one port if the device has more than one lumen. The port can be sealed following filling the lumen with the fill composition.

A body member including a lumen can include one or more apertures from which the bioactive agent can be released. For example, the body member may include a plurality of apertures. The apertures can be present in the wall of the body member in a random or ordered arrangement.

In some aspects, the device includes a lumen and a bioactive agent that can be released from the lumen. As an alternative to a coating, or in addition to a coating, the device can include a lumen filled with bioactive agent and a bioactive agent control-release component(s). Exemplary control-release component(s) that can be used to fill the lumen include polymeric materials. Polymeric materials can be used to form a bioactive agent containing “matrix” in the lumen, from which bioactive agent can be released. A polymeric matrix will refer to herein a body of polymeric material that is associated with the device, such as being present in the lumen, and that is in a form other than a coating. Polymeric materials, such as those described herein, can be used to form a coating or to fill a lumen.

The implantable ocular device can be sterilized utilizing common sterilization techniques, prior to implantation into the body. Sterilization can be accomplished, for example, utilizing ethylene oxide or gamma sterilization, as desired. In preferred embodiments, sterilization techniques utilized do not affect the polymeric coated composition (for example, by affecting release of the bioactive agent, stability of the coating, and the like). The sterilized device can be placed in a package to maintain sterility prior to use.

The term “implantation site” refers to the ocular site at which the implantable ocular device is placed according to the invention. In turn, a “treatment site” includes the implantation site as well as the ocular area that is to receive treatment directly or indirectly from a device component. For example, bioactive agent can migrate from the implantation site to areas surrounding the device itself, thereby treating a larger area than simply the implantation site.

The device can be designed for insertion through a small puncture or incision in the eye that requires few or no sutures for scleral closure at the conclusion of the surgical procedure.

Insertion of the implantable ocular device can be facilitated using an insertion instrument. Examples of suitable instruments for the insertion of coil-shaped device are described in copending and commonly assigned U.S. Pub No. 2007/0027452 (Varner et al.) and U.S. Provisional Patent Application No. 61/247,127 entitled CARRIER FOR AN INSERTABLE MEDICAL DEVICE, INSERTION INSTRUMENTS, AND METHODS OF USE, filed Sep. 30, 2009 (Zhou, J. et al.). The insertion instruments described in this publication includes hand-held devices which can be operated manually or automatically to provide rotational insertion of the device into the eye.

In some cases, the implantable ocular device can be preloaded in the insertion instrument, which can expedite the implantation procedure. A preloaded insertion instrument can be provided in a sterile package. In other cases, the implantable ocular device can be provided in a sterile package and then loaded into the instrument prior to the insertion procedure. The sterile package can include a feature that facilitates mounting of the device on the instrument. The invention contemplates kits including an insertion instrument with preloaded device or a packaged implantable ocular device with a mounting assist (alone or in combination with an insertion instrument). An exemplary kit or system that includes a preloaded implantable ocular device is described in U.S. Provisional Patent Application No. 61/247,127 (supra).

The insertion instrument of the present invention can be used in a method for rotatably inserting the ocular device into the eye. The coil shape of the body member allows the device to be screwed or twisted into the eye, through an insertion in a portion of the eye, such as the sclera. The insertion can be approximately the same size as the outer diameter of the body member. Typical insertion procedures involve advancing the distal portion of the device by rotational movement into the vitreous of the eye. In many cases, in order for the coil shaped body member to be placed into the vitreous, the distal end is first advanced through a scleral region, or scleral and conjunctival regions of the eye. In some aspects, the device can be driven through the scleral tissue through a penetration in the scleral tissue (trans-scleral insertion) caused by a sharp distal end of the device. Alternatively, in other aspects, the device can be driven into the vitreous through a sclerotomy previously made in the eye.

The insertion instrument can include a distal portion that is able to hold the implantable ocular device during the insertion process. In particular one end of the insertion instrument can include a collet-type member that grips a portion of the cap of the implantable ocular device, leaving the coiled portion of the device with the sharpened distal end pointed towards the insertion site, and free from contact with the distal end of the device. The collet-type member can contract and expand radially to grasp and release the cap portion of the implantable ocular device. The collet-type member can also be controlled by an actuator on the insertion instrument.

In many aspects of the invention the distal end of the body member is sharpened or pointed to pierce the scleral tissue during implantation of the device into the eye (in these aspects separate equipment and/or procedures for making an incision or penetration is not required). In an insertion process, the device is held in place using the insertion tool and the distal end is placed in contact with the sclera. Force is then applied to drive the device towards the eye, as well as rotational force. Upon application of these forces, the sharpened point on the distal end pierces the scleral tissue, and the distal portion of the device begins to moves through the scleral tissue. The sharpened distal end, in addition to the inventive configuration of the first portion (having a pitch that is greater than the second portion) facilitates the penetration and movement of the distal portion of the device into the scleral tissue, and significantly minimizes damage to the tissue as well.

During the insertion process, the device is rotated in a direction that causes movement of the coiled body member through the sclera and into the vitreous.

The first portion of the body member (with the extended pitch) is the initial part of the device to move through the scleral tissue and into the vitreous. Because of the extended pitch, there is greater movement in the proximal to distal direction upon a single rotation of the device.

Next, the second portion body member of the device (having a pitch that is less than the second portion) is the subsequent part of the device to move through the scleral tissue. Because the pitch of the second portion is less than the first portion, the gaps or spacings between the “rings” of the body member are smaller, resulting in a tighter fit with the scleral layer. Due to the tighter fit, a small increase in the resistance to rotation can be experienced, and which may require slightly more rotational force to drive the device into the eye.

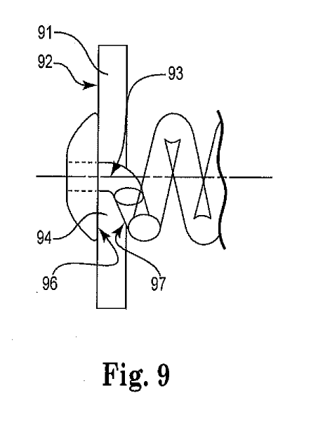

Referring now to FIG. 9, the device is rotated through the layer of scleral tissue 91 to a point wherein the distal face 96 of the cap contacts the outer surface of the eye 92. At this point the device is fully inserted into the eye. The majority of the coiled part of the device (including the first and second portions of the coiled body member) resides in contact with the vitreal fluid. When fully inserted, the transitional portion 93 traverses the layer of scleral tissue. Further, upon full insertion a part of the scleral tissue 94 becomes wedged between the distal face of the cap and a surface 97 of the body member near the transitional portion. The wedging of the scleral tissue between the cap and the body member helps stabilize the device in its fully insertion position and help reduce movement of the device.

Furthermore, in some embodiments, the distal face of the cap has a slightly concave shape, which also improves stabilization of the device when fully inserted in the eye. The concave shape of the distal face provides increased contact with the outer surface of the eye, which has a convex shape. The increased contact minimizes unwanted movement of the device when fully inserted by hindering rocking of the device on the eye surface.

After the device has been fully inserted into the eye using the insertion tool, the collet-type member can be actuated to release the cap portion from its grip, thereby freeing the device.

Optionally, other surgical methods or instruments can be used for implantation of the device. In some methods for inserting the device, an incision in the sclera is made to provide access to the eye. Conventional techniques can be used for the creation of the sclerotomy. Referring to FIG. 1, such techniques include the dissection of the conjunctiva 6 and the creation of pars plana scleral incisions through the sclera 5. The dissection of the conjunctiva 6 typically involves pulling back the conjunctiva 6 about the eye so as to expose large areas of the sclera 5, and the clipping or securing of the conjunctiva 6 in that pulled back state (the normal position of the conjunctiva is shown in phantom). In other words, the sclera 5 is exposed only in the areas where the pars plana scleral incisions are to be made. The device is then inserted through this incision. Thus, the incision should be made large enough to accommodate the device. The conjunctiva will be returned to cover the devices and sutured.

Alternatively, the creation of the sclerotomy can be accomplished by use of an alignment device and method, such as that described in U.S. Pat. No. 7,077,848, that enables sutureless surgical methods and devices thereof. In particular, such methods and devices do not require the use of sutures to seal the openings through which devices are inserted. The alignment devices are inserted through the conjunctiva and sclera to form one or more entry apertures. Exemplary alignment devices are metal or polyimide cannulas through which the devices are inserted into the eye.

After the device is fully inserted into the eye, it can be left there for a period of time so that bioactive agent is released from the device into the vitreous for the treatment of an ocular condition.

The term “treatment course” refers to the dosage rate over time of one or more bioactive agents, to provide a therapeutically effective amount for the treatment of the ocular condition. Thus, factors of a treatment course include dosage rate and time course of treatment (total time during which the bioactive agent(s) is administered).

As used herein, “therapeutically effective amount” refers to that amount of a bioactive agent alone, or together with other substances, that produces the desired effect (such as treatment of an ocular condition such as an ocular disease or the like, or alleviation of pain) in a patient. During treatment, the therapeutically effective amount can depend upon factors such as the particular condition being treated, the severity of the condition, the individual patient parameters including age, physical condition, size and weight, the duration of the treatment, the nature of the particular bioactive agent thereof employed and the concurrent therapy (if any), and like factors within the knowledge and expertise of the health practitioner. A physician or veterinarian of ordinary skill can readily determine and prescribe the effective amount of the bioactive agent required to treat and/or prevent the progress of the condition.

The bioactive agent can be released for a period of time and in an amount sufficient to treat an ocular condition in a subject. In some aspects, the device includes a coating and the bioactive agents can be released from the coating at a steady rate, meaning that there is not substantial variation in amount of bioactive agent released per day over the bioactive agent release period from the coating. Given this, a coatings on the device can allow for drug delivery that is close to a zero-order release rate.

In some aspects, the bioactive agent is released at an average rate in the range of 10 ng/day to 10 μg/day. In more specific aspects, the bioactive agent is released at an average rate in the range of 100 ng/day to 7.5 μg/day. In yet more specific aspects, the bioactive agent is released at an average rate in the range of 500 ng/day to 5 μg/day. In yet more specific aspects, the bioactive agent is released at an average rate in the range of 750 ng/day to 2.5 μg/day. In yet more specific aspects, the bioactive agent is released at an average rate of approximately 1 μg/day.

Coatings can be prepared having a particularly long bioactive agent release period, in which therapeutically effective amounts of bioactive agent are able to be released at later points during this period. With regard to bioactive agent release, the coating can have a “half-life,” which is the period of time at which half of the total amount of bioactive agent that is present in the coating is released.

For example, in one aspect, 50% of the amount of bioactive agent present in the coating is released from the coating after a period of 100 days. In this regard, the coating can be used for the treatment of medical conditions wherein bioactive agent is to be released for a period of time of about 3 months or greater, a period of time of about 6 months or greater, a period of time of about 9 months or greater, a period of time of about 12 months or greater, a period of time in the range of about 3 to about 6 months, a period of time in the range of about 3 to about 9 months, a period of time in the range of about 3 to about 12 months, or a period of time in the range of about 3 to about 24 months.

Once the bioactive agent has been delivered to the implantation site, the implantable ocular device can be removed if the required therapeutically effective amount of bioactive agent has been delivered for treatment of the condition.

The implantable ocular device can provides the ability to deliver one or more bioactive agents in a controlled release manner. As used herein, “controlled release” refers to release of a compound (for example, a bioactive agent) into a patient's body at a desired dosage (including dosage rate and total dosage) and duration of treatment. For example, a coating composition (including the amounts and ratios of the individual components in the coating composition) can be prepared to provide a coating having a desired release profile (amount of bioactive agent released from the coating per unit time) of the bioactive agent. The release kinetics of the bioactive agent in vivo may include both a short term (“burst”) release component, within the order of minutes to hours or less after implantation of the device, and a longer term release component, which can range from on the order of hours to days or even months of useful release. The acceleration or deceleration of bioactive agent release can include either or both of these release kinetics components.

The desired release profile of the bioactive agent can depend upon such factors as the particular bioactive agent selected, the number of individual bioactive agents to be provided to the implantation site, the therapeutic effect to be achieved, the duration of the device in the eye, and other factors known to those skilled in the art.

The ability to provide controlled release of a bioactive agent from the device in the eye can provide many advantages. For example, the implantable ocular device can be maintained in the eye for any desired amount of time, and the release kinetics of the bioactive agent can be adjusted to deliver the total amount of bioactive agent, at the desired rate, to achieve a desired therapeutic effect. In some embodiments, the ability to provide controlled release of bioactive agent in the eye allows implantation of only one device, which can be maintained in place until the desired therapeutic effect is achieved, without need to remove the device and replace the device with a new supply of bioactive agent. Use of the implantable ocular device can, in some aspects, circumvent the need for systemic application of bioactive agents, which can harm other tissues of the body.

The implantable ocular device can be utilized to deliver any desired bioactive agent or combination of bioactive agents to the eye, such as the bioactive agents described herein. The amount of bioactive agent(s) delivered over time is desirably within the therapeutic level, and below the toxic level. For example, a target dosage for triamcinolone acetonide for use in treating diseases or disorders of the eye is in the range of about 0.5 μg/day to about 2 μg per day. The treatment course can be greater than 6 months, or greater than one year. Thus, in some embodiments, the bioactive agent is released from a coating in a therapeutically effective amount for a period of 6 months or more, or 9 months or more, or 12 months or more, or 36 months or more, when implanted in a patient.

Embodiments of the invention provide an implantable ocular device that can release bioactive agent at a constant rate over extended periods of time. Moreover, the implantable ocular device can provide the ability to control the rate of release of bioactive agent by altering the formulation of the coating composition (for example, by providing a first polymer and a second polymer in different relative amounts, and/or by altering the amount of bioactive agent included in the coating composition). Coated compositions described herein can provide release of a bioactive agent in a reproducible manner, for varying time periods, over a range of release rates. Some coating compositions have varying amounts of poly(ethylene-co-vinyl acetate) relative to the amount of poly(n-butyl)methacrylate, and a constant amount of a bioactive agent. In various embodiments, the polymer composition of the coating compositions can be manipulated to control the release rate of the bioactive agent.

The implantable ocular device can be used to deliver one or more bioactive agents to the eye for the treatment of a variety of ocular conditions such as, for example, retinal detachment; occlusions; proliferative retinopathy; proliferative vitreoretinopathy; diabetic retinopathy; inflammations such as uveitis, choroiditis, and retinitis; degenerative disease (such as age-related macular degeneration, also referred to as AMD); vascular diseases; and various tumors including neoplasms. In yet further embodiments, the implantable ocular device can be used post-operatively, for example, as a treatment to reduce or avoid potential complications that can arise from ocular surgery. In one such embodiment, the implantable ocular device can be provided to a patient after cataract surgical procedures, to assist in managing (for example, reducing or avoiding) post-operative inflammation.

In some modes of practice, a bioactive agent is released from a coating formed on the surface of the body member of the device and is used to treat an ocular condition. In another mode of practice, a bioactive agent is released from a lumen within the body member of the device and is used to treat an ocular condition.

In some modes of practice, the implantable ocular device is used for the treatment of diabetic retinopathy, which is characterized by angiogenesis in the retinal tissue.

Diabetic retinopathy has four stages. While the implantable ocular device can be delivered to a subject diagnosed with diabetic retinopathy during any of these four stages, it is common to treat the condition at a later stage.

The first stage is mild nonproliferative retinopathy which is characterized by the appearance of microaneurysms in retinal blood vessels. The second stage is moderate nonproliferative retinopathy which is characterized by blockage of the retinal blood vessels. The third stage is severe nonproliferative retinopathy which is characterized by a more extensive blockage of the retinal blood vessels, which deprive several areas of the retina with their blood supply and results in the formation of new blood vessels in the retina (angiogenesis) in response to this deprivation. The fourth stage is proliferative retinopathy which is characterized by active formation of new blood vessels, which have an abnormal morphology. These abnormally-formed vessels grow along the retinal and vitreal surface and are prone to leak blood, which can result in severe vision loss.

The treatment of diabetic retinopathy can be accomplished by providing an implantable ocular device comprising a bioactive agent that is an anti-angiogenic factor, inserting the device into the eye, and allowing the anti-angiogenic factor to be released from the device. The anti-angiogenic factor can affect the sub-retinal tissue during the treatment course. In some aspects the bioactive agent is an inhibitor of angiogenesis such as anecortave acetate, or a receptor tyrosine kinase antagonist.

Compounds and methods for treating diabetic retinopathy with a receptor tyrosine kinase antagonist have been described in U.S. Pat. No. 5,919,813 (also described herein). Exemplary dosage ranges using a compound of formula I are from about 1 mg/kg/day to about 100 mg/kg/day, or more specifically from about 15 mg/kg/day to about 50 mg/kg/day.

Combination drug delivery strategies can also be carried out for the treatment of diabetic retinopathy. For example, retinal tissue can be treated with one or more neurotrophic factors. In addition, neuroprotective agents can be delivered from the implantable ocular device. As an example, minocycline is thought to be a neuroprotective agent (in addition to its role as an antibiotic with anti-inflammatory effects) as it may also prevent the cascade of events leading to programmed cell death (apoptosis).

The treatment of diabetic retinopathy can be performed by implantation of the implantable ocular device alone, or can be performed with other procedures such as laser surgery and/or vitrectomy.

The implantable ocular device can also be used for the treatment of uveitis, which is characterized by inflammation of the uvea. The uvea is the layer of the eye between the sclera and the retina and includes the iris, ciliary body, and choroid. The uvea provides most of the blood supply to the retina.

Forms of uveitis include anterior uveitis, which typically involves inflammation that is limited to the iris (iritis). Another form of uveitis involves inflammation of the pars plana (between the iris and the choroid). Another form of uveitis is posterior uveitis affects primarily the choroid (choroiditis). The implantable ocular device can be delivered to a target site in the eye for the treatment of any of these particular conditions.

The implantable ocular device can be used to treat uveitis by delivering one or more anti-inflammatory factors to the eye.

The ocular device can also be used for the treatment of retinitis pigmentosa, which is characterized by retinal degeneration. For example, the implantable ocular device can be used to treat retinitis pigmentosa by delivering one or more neurotrophic factors to the eye.

The implantable ocular device can also be used for the treatment of age-related macular degeneration (AMD). AMD is characterized by both angiogenesis and retinal degeneration. Specific forms of AMD include, but are not limited to, dry age-related macular degeneration, exudative age-related macular degeneration, and myopic degeneration. The implantable ocular device can be implanted in the eye for the treatment of any of these forms of AMD. As an example, the implantable ocular device can be used to deliver one or more of the following drugs for the treatment of AMD: anti-VEGF (vascular endothelial growth factor) compounds, neurotrophic factors, and/or anti-angiogenic factors. In some specific aspects, the implantable ocular device is used to release a corticosteriod for the treatment of sub-retinal tissue.

In an exemplary embodiment, the dosage of the steroid is between about 10 μg and about 500 μg over a period of time in the range of about three to about twelve months. This dosage range is applicable to each of the three following stages of macular degeneration, namely: early onset macular degeneration, atrophic macular degeneration (AMD) and neovascular macular degeneration (NMD).

The implantable ocular device can also be used for the treatment of glaucoma, which is characterized by increased ocular pressure and loss of retinal ganglion cells. The implantable ocular device can be implanted in the eye for the treatment of glaucoma contemplated for the release of one or more neuroprotective agents that protect cells from excitotoxic damage. Such agents include N-methyl-D-aspartate (NMDA) antagonists, cytokines, and neurotrophic factors.

The implantable ocular device can also be used for the prophylactic treatment of a subject. In other words, the implantable ocular device may be provided to a subject even if there has not been a diagnosed existence of a disorder or disease. For example, in more than 50% of cases where AMD occurs in one eye, it will subsequently occur in the unaffected eye within a year. In such cases, prophylactic administration of a therapeutic medium such as a steroid into the unaffected eye may prove to be useful in minimizing the risk of, or preventing, AMD in the unaffected eye.

What is claimed is:

1. An ocular drug delivery device comprising,
a proximal portion configured to contact the sclera of the eye, and
a distal portion comprising a coil-shaped body member,
wherein the coil-shaped body member comprises a first portion and a second portion, wherein first portion has a pitch that is greater than the second portion, and the second portion is proximal to the first portion, and
wherein the device comprises a bioactive agent.

2. The ocular drug delivery device of claim 1 wherein the second portion has a length that is greater than a length of the first portion.

3. The ocular drug delivery device of claim 1, wherein the second portion has a length in the range of 2.12 mm to 3.53 mm.

4. The ocular drug delivery device of claim 1, wherein the second portion comprises two to four full rotations.

5. The ocular drug delivery device of claim 1, wherein the first portion has a length in the range of 1.28 mm to 2.14 mm.

6. The ocular drug delivery device of claim 1, wherein the second portion has a pitch in the range of 0.74 mm to 1.23 mm.

7. The ocular drug delivery device of claim 1, wherein the first portion has a pitch in the range of 1.2 mm to 2 mm.

8. The ocular drug delivery device of claim 1 wherein the coil-shaped body member comprises three to five full rotations.

9. The ocular drug delivery device of claim 1 which has a length in the range of 4.9 mm to 6.5 mm.

10. The ocular drug delivery device of claim 1 which has an outer diameter in the range of 1.28 mm to 2.19 min.

11. The ocular drug delivery device of claim 1 wherein the coil-shaped body member has a surface area in the range of 18.9 mm² to 49.5 mm².

12. The ocular drug delivery device of claim 1 wherein the proximal portion comprises a cap having a distal face which contacts the outer surface of the eye when the coil-shaped body member is inserted into the vitreous.

13. The ocular drug delivery device of claim 12 comprising a transitional portion which is present between the distal face of the cap and a proximal end of the second portion of the coil-shaped body member, wherein the transitional portion is configured to wedge scleral tissue between the distal face of the cap and a surface of the coil-shaped portion of the body member that is proximal to and opposite the distal face of the cap.

14. The ocular drug delivery device of claim 13 wherein the length of the transitional portion is in the range of 0.15 mm to 0.3 mm.

15. The ocular drug delivery device of claim 13 wherein the distance between the distal face of the cap and an outermost surface of a first proximal rotation of the coil-shaped body member is in the range of about 0.5 mm to about 0.65 mm.

16. The ocular drug delivery device of claim 12 wherein the cap comprises a peripheral edge that is rounded.

17. The ocular drug delivery device of claim 12 wherein the cap has a circumference in the range of 4.52 mm to 7.54 mm.

18. The ocular drug delivery device of claim 12 wherein the cap has a thickness in the range of 0.25 mm to 0.64 mm.

19. The ocular drug delivery device of claim 1 wherein a surface of the coil shaped body member comprises a polymeric coating which controls release of the bioactive agent when the device is inserted in the eye.

20. An ocular drug delivery device comprising
a distal portion having a coil-shaped body member,
a proximal portion configured to contact the sclera of the eye, the proximal portion comprising a cap having a distal face which contacts the outer surface of the eye when the coil-shaped body member is inserted into the vitreous, wherein the distal face comprises a concave shape, and
a bioactive agent.

21. An ocular drug delivery device comprising a
a distal portion comprising a coil-shaped body member,
a proximal portion comprising a cap having a distal face which contacts the outer surface of the eye when the coil-shaped body member is inserted into the vitreous,
a transitional portion which is present between the distal face of the cap and a proximal end of the second portion of the coil-shaped body member, wherein the transitional portion is configured to wedge scleral tissue between the distal face of the cap and a surface of the coil-shaped portion of the body member that is proximal to and opposite the distal face of the cap, and a bioactive agent.
